(19) United States
(12) Patent Application Publication
(10) Pub. No.: US 2025/0134505 A1
(43) Pub. Date: May 1, 2025

(54) MEDICAL DEVICE

(71) Applicant: Arsenal Medical, Inc., Watertown, MA (US)

(72) Inventors: Dustin Rabideau, Cambridge, MA (US); Lee Core, Needham, MA (US); Jonathan Merlo, Needham, MA (US); Michael Forniciari, Watertown, MA (US)

(21) Appl. No.: 18/437,804

(22) Filed: Feb. 9, 2024

Related U.S. Application Data
(60) Provisional application No. 63/594,257, filed on Oct. 30, 2023.

(51) Int. Cl. A61B 17/00

(52) U.S. Cl. CPC A61B 17/00491; A61B 2017/00495

(57) ABSTRACT

In one aspect, a medical device is disclosed. The medical device includes a first chamber containing a first composition. The first chamber is connected to a first valve having a first burstable membrane. The medical device includes a second chamber containing a second composition. The second chamber is connected to a second valve having a second burstable membrane. The medical device includes a converger connected to the first valve of the first chamber and the second valve of the second chamber. The converger is configured to mix the first composition and the second composition to produce a reactive mixture.

### CROSS-REFERENCE TO RELATED APPLICATIONS

This patent application is a U.S. Nonprovisional Application which claims priority to and the benefit of U.S. Provisional Application No. 63/594,257, filed Oct. 30, 2023, the contents of which are incorporated herein by reference in their entirety.

### TECHNICAL FIELD

The present disclosure is related to devices. More particularly, the present disclosure is related to a medical device with a burstable membrane.

### BACKGROUND

Modern medical devices can have one or more valve assemblies that may separate compositions from one another. A medical device with burstable valve membrane is described herein.

### SUMMARY

In one aspect, a medical device is disclosed. The medical device includes a first chamber containing a first composition. The first chamber is connected to a first valve having a first burstable membrane. The medical device includes a second chamber containing a second composition. The second chamber is connected to a second valve having a second burstable membrane. The medical device includes a converger connected to the first valve of the first chamber and the second valve of the second chamber. The converger is configured to mix the first composition and the second composition to produce a reactive mixture that forms into a solid.

In another aspect, a method for producing a reactive mixture that forms into a solid is disclosed. The method includes receiving, at a mixing mechanism, a first composition from a first chamber. The first chamber is connected to the mixing mechanism through a first valve having a first burstable membrane. The method includes receiving, at the mixing mechanism, a second composition from a second chamber, wherein the second chamber is connected to the mixing mechanism through a second valve having a second burstable membrane. The method includes mixing, at the mixing mechanism, the first composition with the second composition to produce a reactive mixture. The method includes delivering, by a nozzle connected to the mixing mechanism, the reactive mixture to a patient.

### BRIEF DESCRIPTION OF THE DRAWINGS

FIGS. 1A-B illustrate exemplary embodiments of a rear and front view of a medical device;

FIG. 2 illustrates an exemplary embodiment of a valve;

FIG. 3 illustrates an exemplary embodiment of a valve system; and

FIG. 4 is a table showing various burst pressures of a burstable membrane.

### DETAILED DESCRIPTION

This disclosure presents various devices and methods related to burstable membranes of valves of medical devices. Aspects of the present disclosure may be used to prevent reaction or degradation of components of a medical device from a gas source. In some embodiments, aspects of the present disclosure may be used to prevent polyurea buildup in a medical device. In an embodiment, aspects of the present disclosure may allow for a medical device to utilize burstable membranes while retaining functionality of a check valve.

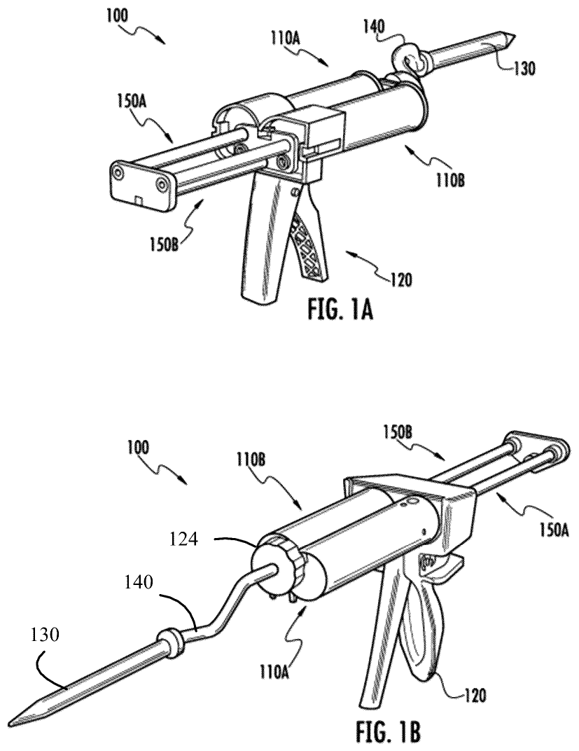

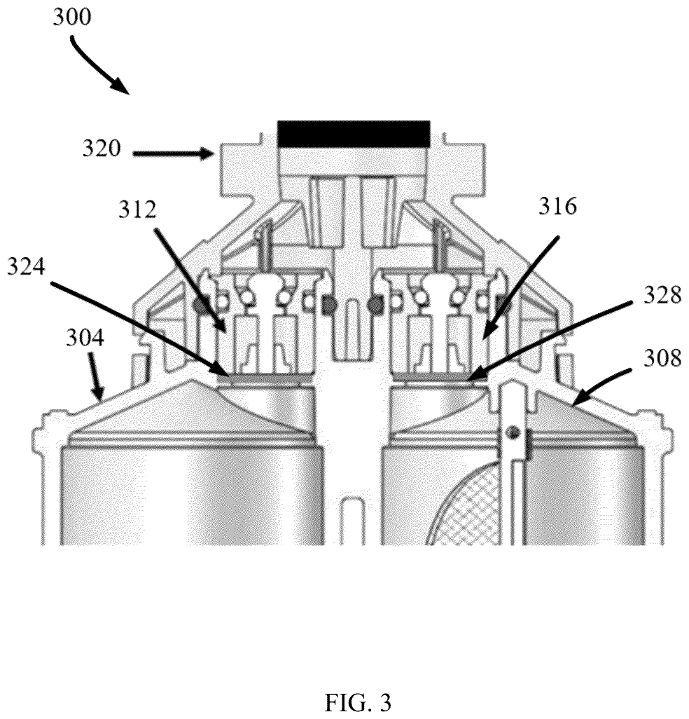

FIG. 1A shows a rear view of exemplary embodiment of a medical device 100. The medical device 100 may have a first chamber 110A. The first chamber 110A may be configured to contain a first composition. A first composition may include one or more chemicals, elements, and the like. A first composition may include a polyol. A first composition may be as described below with reference to FIG. 3. In some embodiments, the second chamber 110B may be configured to contain and/or hold a second composition. A second composition may be the same as a first composition of the first chamber 110A. In other embodiments, a second composition may be different to a first composition of the first chamber 110A. A second composition of the second chamber 110B may include an isocyanate. A second composition of the second chamber 110B may be as described below with reference to FIG. 3.

In some embodiments, the first chamber 110A may have a volume of about 500 ml, without limitation. The first chamber 110A may have a volume of about 50 ml to about 100 ml. In an embodiment, the first chamber 110A may have a volume of about 65 mL. The first chamber 110A may be placed adjacent to the second chamber 110B. The second chamber 110B may be similarly sized as the first chamber 110A. In other embodiments, the second chamber 110B may be smaller or larger than the first chamber 110A. In some embodiments, the first chamber 110A and/or the second chamber 110B may be pressurized. In other embodiments, the first chamber 110A and/or the second chamber 110B may be unpressurized. The first chamber 110A and/or the second chamber 110B may be secured in position through a housing. For instance, and without limitation, a housing may be shaped to receive both an end of the first chamber 110A and an end of the second chamber 110B. A housing of the medical device 100 may have a connecting portion that may connect the flexible connector 140 to the housing. A housing of the medical device 100 may connect nozzle 130 to one or more ends of the first chamber 110A and the second chamber 110B. The nozzle 130 may connect to the first chamber 110A and/or the second chamber 110B through flexible connector 140.

The medical device 100 may include a squeezable grip 120. The squeezable grip 120 may have an ergonomic design which may help facilitate operation by a user. The squeezable grip 120 may include a textured surface. For instance, and without limitation, the squeezable grip 120 may include a rough surface, which may increase a force of friction for a user's grasp. The squeezable grip 120 may have one or more indents, bends, and the like, which may assist in a user's grasp of the squeezable grip 120. The squeezable grip 120 may be positioned underneath a connecting portion of a housing of the medical device 100, for instance a portion of the housing that receives an end of the first chamber 110A and the second chamber 110B. The squeezable grip 120 may be positioned under a center of gravity of the medical device 100 which may allow a user to operate the medical device 100 more efficiently. The squeezable grip 120 may have a static end and a moving end. A static end may be configured to stay in a position while a moving end of the squeezable grip 120 may be configured to move from an extended position towards the static end. For instance, and without limitation, a moving end of the squeezable grip 120 may be angled about 45 degrees away from a static end and may be configured to rotate about an x-axis towards the static end.

The medical device 100 may include a nozzle 130. The nozzle 130 may be configured to output a liquid material or other substance from the first chamber 110A and/or the second chamber 110B. In some embodiments, the nozzle 130 may include a static mixing nozzle. For instance, the nozzle 130 may be configured to mix a first composition of the first chamber 110A with a second composition of the second chamber 110B. Mixing may include a physical merging between two or more substances/compositions. The nozzle 120 may be connected to a housing of the medical device 100 with a flexible connector 140.

The nozzle 130 may be connected to a converger 124 of the medical device 100. The converger 124 of the medical device 100 may be configured to mix a chemical and/or composition of the first chamber 110A and the second chamber 110B. The converger 124 may include a fluidic outlet connecting an end of the first chamber 110A and an end of the second chamber 110B into a single lumen. The converger 124 may, in some embodiments, be connected to and/or include a mixing device. A mixing device may include, without limitation, an impeller or other device. In some embodiments, the nozzle 130 may be configured to mix chemicals and/or compositions of the first chamber 110A and the second chamber 110B. The medical device 100 may be as described in U.S. Pat. No. 10,687,795, filed Jan. 20, 2017, and titled “DELIVERY SYSTEM FOR IN SITU FORMING FOAMS AND METHODS OF USING THE SAME”, which is incorporated by reference herein in its entirety.

In some embodiments, the medical device 100 may include piston push rods 150A,B. The piston push rods 150A,B, may be connected to plungers or other devices of an interior of the first chamber 110A and/or the second chamber 110B. The piston push rods 150A,B, may push compositions from each of the barrels 110A, B. In some embodiments, as the grip 120 is squeezed, it engages the piston push rods 150A, B, through the use, for example, of two friction plate and springs and ratchets the rod forward thereby deploying the material. Other means for engaging the push rods include toothed surfaces, pulleys, and lead screws.

Still referring to FIG. 1A, the nozzle 130 may be configured to deliver a reactive mixture that forms into a solid to a patient. A reactive mixture may include one or more chemicals, compounds, and the like. In some embodiments, a reactive mixture may be expandable. For instance, a reactive mixture may expand to occupy more space than an original size of the reactive mixture. A reactive mixture may be made from a mixing of a chemical and/or composition of the first chamber 110A and the second chamber 110B. In some embodiments, a reactive mixture may be a non-polymer material, such as a solution, suspension, colloid, or biologic mixture. In some embodiments, a reactive mixture delivered from the nozzle 130 may include a polymer foam. A “polymer foam” refers to an article comprising a plurality of cells (i.e. volumes) that are at least partially surrounded by a material comprising a polymer. The cells within the foam may be open or closed. The cells within a polymer foam may be any suitable size. In some embodiments, a polymer foam may comprise at least 10 cells, at least 100 cells, at least 1,000 cells, at least 10,000 cells, or more, without limitation. A polymer foam may have a polymer material. A polymer material may comprise a plurality of polymers which can be, for example, cross-linked to each other in the process of forming a polymer foam. In some embodiments, the polymer material comprises fluid polymers in the substantial absence of a carrier fluid. In other instances, the plurality of polymers in the polymer material are suspended in a carrier fluid (e.g., a liquid suspension medium, etc.). In some embodiments, a polymer may comprise fewer than about 100, fewer than about 50, fewer than about 25, or fewer than about 10 monomer units. In some embodiments, a polymer may comprise between about 2 and about 100, between about 2 and about 50, between about 2 and about 25, between about 5 and about 50, or between about 5 and about 25 monomer units. The polymers within the polymer material can comprise a variety of functional groups that allow the polymers to, for example, cross-link to each other, attach to tissue or other material within a body cavity, interact with agents in the bloodstream of the subject (e.g., imaging agents, cross-linking agents, etc.), among other functionalities. A foam delivered by the nozzle 130 may be as described in U.S. Pat. No. 10,307,515, filed Oct. 30, 2015, and titled “IN SITU FORMING HEMOSTATIC FOAM IMPLANTS”, incorporated by reference herein in its entirety.

In some embodiments, a reactive mixture produced from the nozzle 130 may be applied and/or inserted into a body cavity of an individual. In some embodiments, the polymers within a polymer material may cross-link within a body cavity. The term “cross-linking” is used to refer to the process whereby a pendant group on a first polymer chain may react with a second polymer chain (e.g., a pendant group on the second polymer) or other molecule or molecules to form a covalent or ionic bond joining the two polymers. Polymers that can undergo cross-linking can comprise straight chains, branched chains having one or more arms (i.e., multi-arm chains), or mixtures of these. In some cases, the polymer (branched and/or non-branched) may contain reactive side chains and/or reactive terminal groups (i.e., groups at the end of a polymer chain), and cross-linking may involve reactions between the side chains, between terminal groups, and/or between a side chain and a terminal group.

The medical device 100 may be used in conjunction with site access devices to permit deployment of in situ forming foams into closed cavities, such as the abdominal cavity in cases of internal bleeding, including one or more of the following: abdominal bleeding, junctional/inguinal bleeding, and pelvic bleeding, without limitation. An exemplary procedure to obtain access to the abdominal cavity and insert the delivery system nozzle into the body will include a skin incision and introduction of an entry port into the abdomen directly above the umbilicus. The entry port can have a range of an inner diameter (ID) from 1.8 mm to 13 mm. In other embodiments, the entry port can be less than 1.8 mm or greater than 13 mm in inner diameter. The nozzle 130 may be placed into an entry port which may provide a pathway for a reactive mixture into the cavity. Steps of this exemplary procedure include the following:
(1) Perform a small skin nick with a scalpel above the umbilicus
(2) Insert entry port (such as a Veress needle with dilator sheath) to abdominal cavity
(3) Remove at least a portion of the entry port (e.g. the Veress needle may be retracted), and dispense lubrication into sheath if required.
(4) Advance the nozzle into the abdomen directly, or through the sheath, using a slow rocking motion. An optional positive stop on the delivery nozzle indicates that the nozzle is in the correct location.
(5) Attach nozzle to the delivery system and dispense material.

Devices that may be useful for obtaining access to closed cavities such as the abdominal cavity include, without limitation, a 6 cm Veress needle, a short (e.g. about 5 cm) dilating sheath, or an 11 mm diameter nozzle with a long taper (e.g. about 4.4 cm), without limitation.

Referring now to FIG. 1B, a front view of the medical device 100 is shown. The medical device 100 may be the same as that of FIG. 1A. In some embodiments, the nozzle 130 be connected to the flexible connector 140. The flexible connector 140 may be made of any suitable material, such as, but not limited to, rubber, plastic, and the like. In some embodiments, the flexible connector 140 may be configured to adjust positions in an x, y, and/or z direction. The nozzle 130 may be configured to move positions along with the flexible connector 140. The flexible connector 140 may be connected to converger 124 of the medical device 100. The converger 124 may, in some embodiments, include a fluidic conduit that connects a first end of the first chamber 110A with a second end of the second chamber 110B to an end of the flexible connector 140.

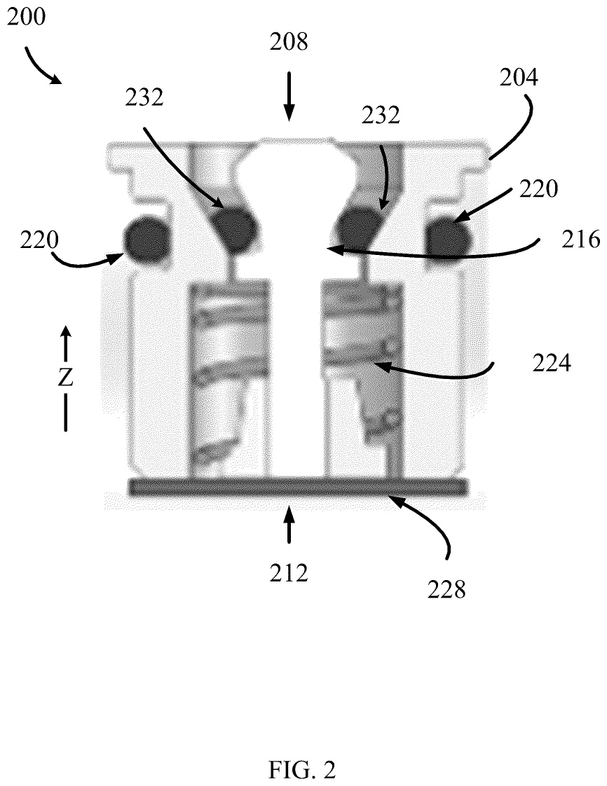

Referring now to FIG. 2, an exemplary embodiment of a cross-sectional view of a burstable valve 200 is illustrated. The burstable valve 200 may include a valve body 204. The valve body 204 may have a width of about 2 inches, greater than 2 inches, or less than 2 inches, without limitation. In some embodiments, the valve body 204 may have a width of about 1 cm. The valve body 204 may have a height of about 3 inches, less than 3 inches, or greater than 3 inches, without limitation. In some embodiments, the valve body may be made of a material such as, but not limited to, metals, plastics, and the like. A material of the valve body 204 may be polypropylene in an embodiment. The valve body 204 may have a first end 208 and a second end 212. The first end 208 may be positioned opposite the second end 212. In some embodiments, the first end 208 may be circular, ovular, or other shapes. The first end 208 may connect to the second end 212 through the valve body 204. For instance, the valve body 204 may have a square/rectangular bottom portion that connects to a circular tube-like portion. The second end 212 may be positioned on a lower rectangular portion of the valve body 204 and the first end 208 may be positioned on an upper circular portion of the valve body 204.

The valve 200 may include poppet 216. The poppet 216 may be made of materials such as, but not limited to, metals, plastics, and the like. The poppet 216 may be made from polypropylene in an embodiment. In some embodiments, the poppet 216 may have a base rod portion connected to an upper head. An upper head of the poppet 216 may be shaped as a diamond, square, circle, polygon, and/or other shapes. A head of the poppet 216 may be adjacent to the first end 208 and a base portion of the poppet 216 may be adjacent to the second end 212. The poppet 216 may be configured to move with a flow of fluid, such as in direction Z. In some embodiments, the poppet 216 may be configured to move opposite direction Z.

The valve 200 may include first ring 220 and second ring 232. The first and second rings 220, 232 may be made of a material such as, but not limited to, rubber, plastic, metal, and the like. The first and second rings 220, 232, may be spherical, ovular, rectangular, or otherwise shaped. The first and second rings 220, 232 may be O-rings, in embodiments. In some embodiments, the first ring 220 may be positioned at a top end of an exterior of the valve body 204. For instance, and without limitation, the first ring 220 may be placed near a neck of the valve body 204. The first ring 220 may mate with and/or seal one or more surfaces of the valve body 204. The first ring 220 may act as a gasket seal that may prevent fluid flow from an exterior of the valve body 204 to an interior of the valve body 204. In some embodiments, the first ring 220 may be located and/or positioned on an outside of the valve body 204. For instance, and without limitation, the first ring 220 may be placed on a circumference of the valve body 204. The first ring 220 may be located on an outside of the valve body 204 and may be configured to prevent an extensive movement of the valve body 204 in the direction Z, without limitation. In some embodiments, the first ring 220 may act as a fluid seal for the valve body 204. The poppet 216 may have straight and/or flat structures on a right and/or left side that may contact the second ring 232. In some embodiments the poppet 216 may be a conical shape. The second ring 232 may mate with and/or fluidically seal the poppet 216. The second ring 232 may act as a gasket seal and may prevent a flow of fluid within the valve body 204. The second ring 232 may create a circumferential seal around the poppet 216. The poppet 216 may be configured to shut off a flow of fluid in an opposite direction of the direction Z. The second ring 232 may be configured to prevent a movement of poppet 216 in the Z direction and/or in a direction opposite of the Z direction. The first and second rings 220, 232, may be configured in any manner that provides a seal to the valve body 204, without limitation.

Still referring to FIG. 2, the valve 200 may include spring 224. The spring 224 may be made of a material such as, but not limited to, copper, aluminum, steel, and the like. The spring 224 may wrap around the poppet 216 in a clockwise or counter-clockwise direction. In some embodiments, the spring 224 may have a spring constant of about, without limitation 5N/M. In other embodiments, the spring 224 may have a spring constant greater than or less than about 5N/M. The spring 224 may have a spring constant of about 1 N/M, in an embodiment. The second ring 232 may be configured to prevent extensive compression or extension of spring 224. The spring 224 may be configured to compress the second ring 232 of the valve body 204. The spring 224 may be configured to compress in the direction Z, in a direction opposite Z, and the like. In some embodiments, the spring 224 may be configured to compress against the poppet 216, such as in the direction Z, in a direction opposite Z, and the like.

The valve 200 may include membrane 228. The membrane 228 may cover and/or seal off the second end 212. In some embodiments, the membrane 228 may cover and/or seal the first end 208. In some embodiments, the membrane 228 may be heat staked to the second end 212. The membrane 228 may include one or more polymers, thin foils, or metals. In some embodiments, the membrane 228 may be made of polypropylene. In some embodiments, the membrane 228 may be made of, without limitation, oriented polymers, foils, multi-layer laminates, and the like. The membrane 228 may be made of a multilayer structure, such as, without limitation, multiple layers of polypropylene or other materials. The membrane 228, in some embodiments, may be configured to be removed through, without limitation, ultrasonic, dissolution, light-absorption/decay, piercing, and/or other film removal methods. The membrane 228 may be about 1 μm to about 12 μm thick. In some embodiments, and without limitation, the membrane 228 may be about 4 μm thick. In some embodiments, the membrane 228 may be less than about 1 μm thick or greater than 10 μm thick. The membrane 228 may be burstable. For instance, under a certain pressure of fluid flowing in the direction Z, the membrane 228 may burst, allowing a flow of the fluid through the valve body 204. The membrane 228 may be configured to burst at about 47 psi. In other embodiments, the membrane 228 may be configured to burst at lower than or higher than about 47 psi, without limitation. A bursting of the membrane 228 may allow for a flow of fluid through the second end 212 to the first end 208. The valve 200 may continue normal operation after a bursting of the membrane 228, in an embodiment and without limitation.

Referring now to FIG. 3, a valve system 300 is presented. The valve system 300 may be implemented in the medical device 100 as described above with reference to FIG. 1A. For instance, and without limitation, the valve system 300 may include first chamber 304 and second chamber 308, which may be the same as the first chamber 110A and the second chamber 110B as described above with reference to FIG. 1.

In some embodiments, the first chamber 304 may contain a first composition. A first composition may include, without limitation, one or more polyols. Polyols may include polyether- and/or polybutadiene-based polyols. Polyols may include polypropylene glycol (PPG) and polyethylene glycol (PEG), as well as random and/or block copolymers thereof. In some embodiments, a first composition of the first chamber 304 may include polycarbonates, polybutadienes, and/or polyesters. Diols, triols, and tetrols may be used, in some embodiments. In some embodiments, multifunctional polyols with any suitable number of arms may be used. Molecular weights between 100 and 10,000 Da are preferable, with molecular weights up to 6,000 Da being most preferred, and blends of polymers with different molecular weights, degrees of branching, and composition are often used. Commercial polymers of particular interest include polypropylene glycols (425, 1200 Da), polyethylene glycols (200, 400, 600, 1000, 2000, 3000 Da), Pluracol products (355, 1135i, 726, 816), Arch Poly-G 30-240, Poly-G 76-120, Poly-G 85-29, trimethylolpropane ethoxylate (450, 1014 Da), pentaerythritol ethoxylate (797 Da), UCON 75-H-1400, UCON 75-H-9500, dipropylene glycol, diethylene glycol, tripropylene glycol, triethylene glycol, tetrapropylene glycol, and tetraethylene glycol. In preferred embodiments, polyols used in the present invention have a polyethylene oxide content of 0-50 wt %, more preferably 0-40 wt %, more preferably 0-30 wt %, more preferably 0-25 wt %, and most preferably 0-16 wt %. Also preferred is that polyols used in the present disclosure comprise an amine catalyst in an amount up to 10 pphp, a water content of up to 20 pphp, a surfactant in an amount up to 10 pphp, and a diluent up to 300 pphp (preferably up to 250 pphp and most preferably up to 15 pphp).

Referring still to FIG. 3, the second chamber 308 may contain a second composition. A second composition of the second chamber 308 may include an isocyanate. Isocyanates suitable for use in such embodiments include any polymeric isocyanate with a degree of functionality greater than 2.0, with the most useful range being 2.0-2.7. In some embodiments, a polymeric isocyanate with a degree of functionality of 2.0 or less may be used. Polymeric isocyanates may be based on methylene diphenyl isocyanate (MDI). Isocyanate true-prepolymers and quasi-prepolymers may also be used. A “quasi-” prepolymer, or semi-prepolymer, is a polymer formed by the reaction between a multifunctional isocyanate and polyol, where the isocyanate-to-alcohol ratio is greater than the stoichiometric two-to-one ratio. A “true-” prepolymer, or strict-prepolymer, is a polymer formed by the reaction between a multifunctional isocyanate and polyol, where the isocyanate-to-alcohol ratio is equal to the stoichiometric two-to-one ratio.

The first chamber 304 may be connected to first valve 312 and the second chamber 308 may be connected to second valve 316. The first valve 312 and/or the second valve 316 may be as described above with reference to FIG. 2. The first valve 312 may include a first burstable membrane 324, such as a polypropylene film. The first burstable membrane 324 of the first valve 312 may act as a filter or shield from substances of the first chamber 304. For instance, the first burstable membrane 324 of the first valve 312 may prevent a gas source of the first chamber 304 from travelling to the converger 320 and vice versa. A gas source may include, without limitation, water vapor, polyol vapor, and the like. Similarly, the second burstable membrane 328 of the second valve 316 may be configured to prevent a gas source of the second chamber 308 from interacting with the converger 320 and vice versa. A gas source of the second chamber 308 may include, without limitation, water vapor, isocyanate vapor, and the like. The first and second burstable membranes 324, 328, may prevent isocyanate and/or polyol from contacting each other and/or ambient water vapor inside the valves 312, 316, and/or converger 320. The first and second burstable membranes 324, 328 may be configured to prevent polyurea buildup within the first valve 312, second valve 316, and/or converger 320. In some embodiments, the first and second burstable membranes 324, 328, may be heat staked to the first valve 312 and/or the second valve 316.

In some embodiments, the first valve 312 and the second valve 316 may be configured to open at about, without limitation, 15 psi, less than 15 psi, or greater than 15 psi of pressure. In some embodiments, the first valve 312 and/or the second valve 316 may be configured to open at about 45 psi. The first valve 312 and the second valve 316 may allow a flow of isocyanate and/or polyol from the first chamber 304 and the second chamber 308 to flow upwards through the first valve 312 and the second valve 316 into the converger 320. The converger 320 may mix a first composition of the first chamber 304 and a second composition of the second chamber 308. In some embodiments, the converger 320 may include an impeller or other mixing device.

Membranes of the first valve 312 and/or the second valve 316 may be compatible with one or more sterilization cycles of the valve system 300. Sterilization cycles may include, without limitation, vacuum pump/purge cycles, gamma, e-beam, ultraviolet, ethylene oxide gas, and/or other sterilization cycles. In some embodiments, the first valve 312 and/or the second valve 316 may be configured to aerate one or more gases, such as ethylene oxide gas.

The converger 320 may mix a first composition with a second composition of the first chamber 304 and the second chamber 308, which may produce an expandable material. In some embodiments, an expandable material may include a foam. A foam may include a polymer foam. In some embodiments, a polymer of a polymer foam comprises a synthetic polymer. As used herein, a “synthetic polymer” refers to a polymer that is a product of a reaction directed by human interaction. For example, synthetic polymers can include polymers synthesized by reactions of natural or synthetic monomers or combinations thereof that are directed by human interaction. The formation of synthetic polymers can also include chain elongation of natural or synthetic polymers. In some embodiments, the synthetic polymer is not found in nature. In other cases, the synthetic polymer can be found in nature, but the polymer is synthesized via human interaction (e.g., in a laboratory setting). In some embodiments, the polymer may comprise a poly alpha-hydroxy acid. In some cases, the polymer may comprise a polyester. In some cases, the polymer may comprise a polyether-polyester block copolymer. In some cases, the polymer may comprise a poly (trimethylene carbonate). In some embodiments, the backbone of the polymer can exclude at least one of polynucleotides, proteins, and polysaccharides.

In some embodiments, a polymer foam is formed by cross-linking a condensation polymer of a polyol and a polyacid. Examples of polyols suitable for use in forming the condensation polymer used to form the polymer foams described herein include, but are not limited to, glycerol, polyethylene glycol, polypropylene glycol, polycaprolactone, vitamin B6, erythritol, threitol, ribitol, arabinitol, xylitol, allitol, altritol, galactritol, sorbitol, mannitol, iditol, lactitol, isomalt, and maltitol, wherein the functional groups present on the polyol are optionally substituted. Examples of polyacids suitable for use in forming the condensation polymer used to form the polymer foams described herein include, but are not limited to, succinic acid, fumaric acid, a-ketoglutaric acid, oxaloacetic acid, malic acid, oxalosuccinic acid, isocitric acid, cis-aconitic acid, citric acid, 2-hydroxy-malonic acid, tartaric acid, ribaric acid, arabanaric acid, xylaric acid, allaric acid, altraric acid, galacteric acid, glucaric acid, mannaric acid, dimercaptosuccinic acid, oxalic acid, malonic acid, succinic acid, glutaric acid, adipic acid, pimelic acid, suberic acid, azelaic acid, sebacic acid, malic acid, or vitamin B6, wherein the functional groups present on the polyacid are optionally substituted.

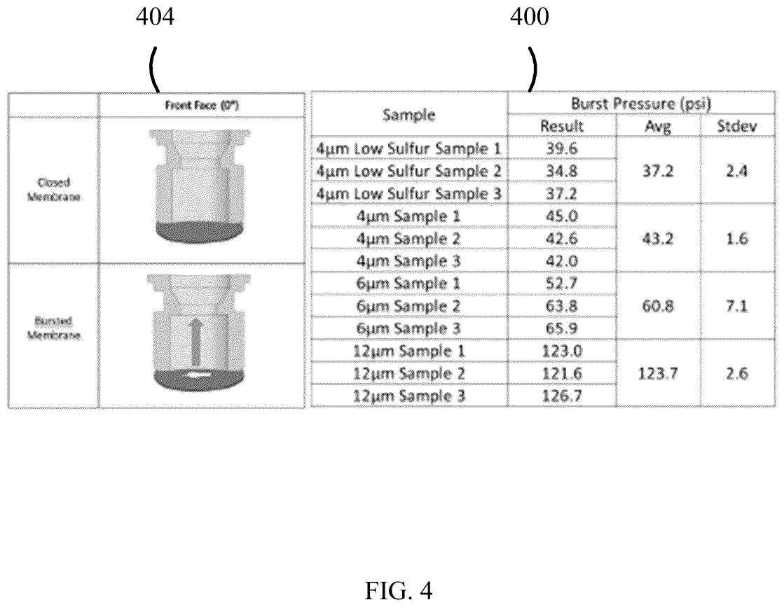

Referring now to FIG. 4, a table 400 showing various burst pressures of various thickness of membranes is shown next to schematics 404. The schematics 404 show a cross-section view of a closed membrane and a burst membrane of a valve. Table 400 shows repeat results of burst pressure testing a 4 μm, 6 μm, and 12 μm polypropylene membrane, as well as a 4 μm low sulfur polypropylene membrane, each with three replicate samples (sample 1, sample 2, and sample 3).

The 4 μm film was tested against the 4 μm low sulfur film. The average burst pressure of the 4 μm film was about 43.2 psi with a standard deviation of about 1.6, while the average burst pressure of the 4 μm low sulfur film was about 37.2 psi with a standard deviation of about 2.4.

A 6 μm film was tested against the 4 μm films. The average burst pressure of the 6 μm film was about 60.8 psi with a standard deviation of about 7.1 psi.

A 12 μm film was tested against the 4 μm and 6 μm films. The 12 μm film had an average burst pressure of about 123.7 psi with a standard deviation of about 2.6 psi.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A medical device, comprising;
a first chamber containing a first composition, wherein the first chamber is connected to a first valve having a first burstable membrane;
a second chamber containing a second composition, wherein the second chamber is connected to a second valve having a second burstable membrane;
a converger connected to the first valve of the first chamber and the second valve of the second chamber, wherein the converger is configured to mix the first composition and the second composition to produce a reactive mixture that forms a solid; and
a nozzle connected to the converger and configured to deliver the reactive mixture material to a patient.

2. The medical device of claim 1, wherein the burstable membrane of the first valve and the second valve are configured to prevent gas diffusion from a gas source to an interior of the converger, or from an interior of the converger to a gas source.

3. The medical device of claim 2, wherein the gas source includes water vapor.

4. The medical device of claim 1, wherein the first composition comprises a polyol.

5. The medical device of claim 1, wherein the second composition comprises an isocyanate.

6. The medical device of claim 1, wherein the burstable membrane of the first valve and the second valve is a polypropylene membrane.

7. The medical device of claim 1, wherein the burstable membrane of the first valve and the second valve is configured to burst at a pressure of about 45 psi.

8. The medical device of claim 1, wherein the burstable membrane of the first valve and the second valve is about 1 μm to about 5 μm thick.

9. The medical device of claim 1, wherein the reactive mixture comprises an elastomeric polyurethane.

10. The medical device of claim 1, wherein the reactive mixture reacts in a body cavity of the patient to generate a gas, thereby forming a polymer formulation.

11. A method of producing a reactive mixture, comprising:
receiving at a mixing mechanism, a first composition from a first chamber, wherein the first chamber is connected to the mixing mechanism through a first valve having a burstable membrane;
receiving, at the mixing mechanism, a second composition from a second chamber, wherein the second chamber is connected to the mixing mechanism through a second valve having a burstable membrane;
mixing, at the mixing mechanism, the first composition with the second composition to produce an reactive mixture; and
delivering, by a nozzle connected to the mixing mechanism, the reactive mixture to a patient.

12. The method of claim 11, wherein the burstable membrane of the first valve and the second valve are configured to prevent gas diffusion from a gas source to an interior of the mixing mechanism or from an interior of the mixing mechanism to a gas source.

13. The method of claim 12, wherein the gas source includes water vapor.

14. The method of claim 11, wherein the first composition comprises a polyol.

15. The method of claim 11, wherein the second composition comprises an isocyanate.

16. The method of claim 11, wherein the burstable membrane of the first valve and the second valve is a polypropylene membrane.

17. The method of claim 11, wherein the burstable membrane of the first valve and the second valve is configured to burst at a pressure of about 45 psi.

18. The method of claim 11, wherein the burstable membrane of the first valve and the second valve is about 1 μm to about 5 μm thick.

19. The method of claim 11, wherein the reactive mixture comprises an elastomeric polyurethane.

20. The method of claim 11, wherein the reactive mixture reacts in a body cavity of the patient to generate a gas, thereby forming a polymer formulation.
